(19) United States
(12) Patent Application Publication
(10) Pub. No.: US 2015/0342480 A1
(43) Pub. Date: Dec. 3, 2015

(54) OPTICAL PULSE-RATE SENSING

(71) Applicant: Microsoft Corporation, Redmond, WA (US)

(72) Inventors: Gregory Kim Justice, Redmond, WA (US); Ryna Karnik, Redmond, WA (US); Daniel C. Canfield, McMinnville, OR (US); Joshua Mark Hudman, Issaquah, WA (US); Gabriel Michael Rask Gassoway, Redmond, WA (US); Vinod L. Hingorani, Redmond, WA (US); Mohammad Shakeri, Kirkland, WA (US)

(21) Appl. No.: 14/292,561

(22) Filed: May 30, 2014

(51) Int. Cl. A61B 5/024; A61B 5/00

(52) U.S. Cl. CPC A61B 5/02427; A61B 5/02438; A61B 5/7225; A61B 2562/185

(57) ABSTRACT

An optical pulse-rate sensor includes a fixture, a light emitter, a light sensor, and a light stop. The fixture has a rim configured to contact a skin surface and enclose an area of the surface. The light emitter and light sensor are each coupled to the fixture and positioned opposite the area. The light stop is coupled to the fixture and positioned between the light emitter and the light sensor to shield the light sensor from direct illumination by the light source.

### BACKGROUND

Measurement of the pulse rate of a human subject is traditionally done in a clinical setting, using dedicated medical equipment. At the present time, however, there is increasing demand for non-clinical pulse-rate sensing, to support athletic and fitness activity, for example. As a result, pulse-rate sensors have been incorporated into wearable consumer devices marketed to athletes and fitness enthusiasts. Various issues arise, however, in adapting medical technology to suit the desires of the consumer. One specific issue is how to miniaturize a pulse-rate sensor so it can be incorporated into a device desirable to be worn. Another issue is how to limit power consumption by the sensor, so that the pulse-rate measurement can track prolonged user activity, such as exercise, without depleting the batteries of the device. A third issue is how to make a reliable a pulse-rate measurement in the presence of everyday noise sources, which may exceed those of a clinical environment.

### SUMMARY

One embodiment of this disclosure provides an optical pulse-rate sensor having a fixture, a light emitter, a light sensor, and a light stop. The fixture includes a rim configured to contact a skin surface and to enclose an area of the surface. The light emitter and light sensor are each coupled to the fixture and positioned opposite the area. The light stop is coupled to the fixture and positioned between the light emitter and the light sensor to shield the light sensor from direct illumination by the light source.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1A schematically shows aspects of an example wearable electronic device.

FIGS. 1B and 1C show additional aspects of an example wearable electronic device.

FIGS. 2A and 2B are exploded views of an example wearable electronic device.

FIG. 3 is an exploded view of a portion of an example wearable electronic device.

FIGS. 4 and 5 are cross-sectional views of an example optical pulse-rate sensor in a wearable electronic device.

FIG. 6 is an isometric view of an example light guide of an optical pulse-rate sensor.

### DETAILED DESCRIPTION

Aspects of this disclosure will now be described by example and with reference to the drawing figures listed above. Components and other elements that may be substantially the same in one or more figures are identified coordinately and described with minimal repetition. It will be noted, however, that elements identified coordinately may also differ to some degree.

This disclosure is directed primarily to an optical pulse-rate sensor that may be incorporated into a wearable electronic device. As described in further detail below, the sensor works by probing the wearer's skin with visible light of wavelengths strongly absorbed by hemoglobin. As the capillaries below the skin fill with blood on each contraction of the heart muscle, more of the probe light is absorbed; as the capillaries empty between contractions, less of the probe light is absorbed. Thus, by measuring the periodic attenuance of the probe light, the wearer's pulse rate can be determined. The pulse-rate sensor described herein includes various features that improve the signal-to-noise ratio of the attenuance measurement, enabling pulse-rate determination in poorly controlled, everyday environments, and using relatively weak probe light for extended battery life.

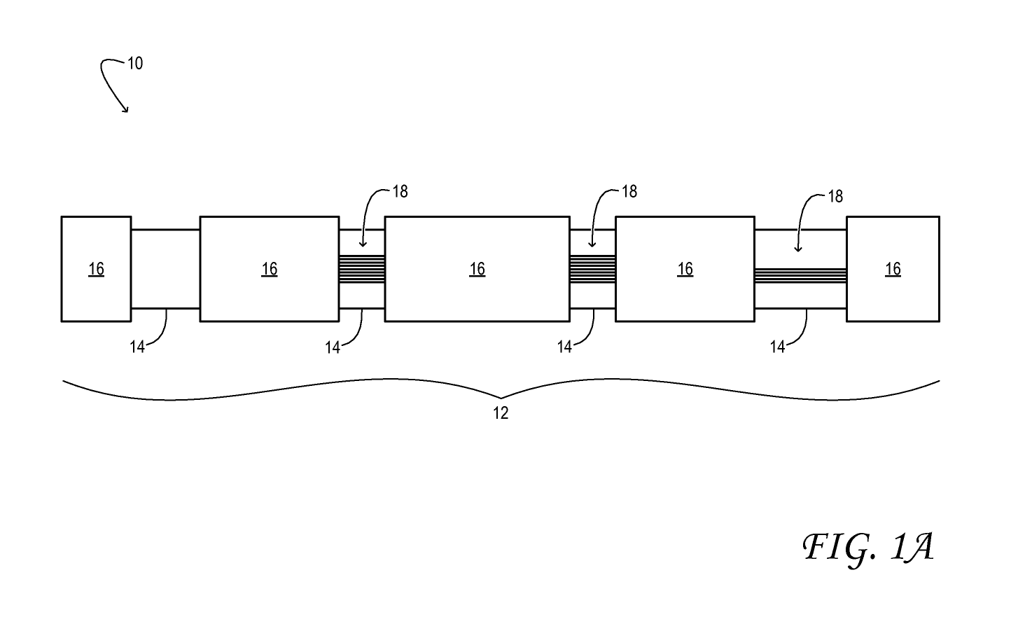

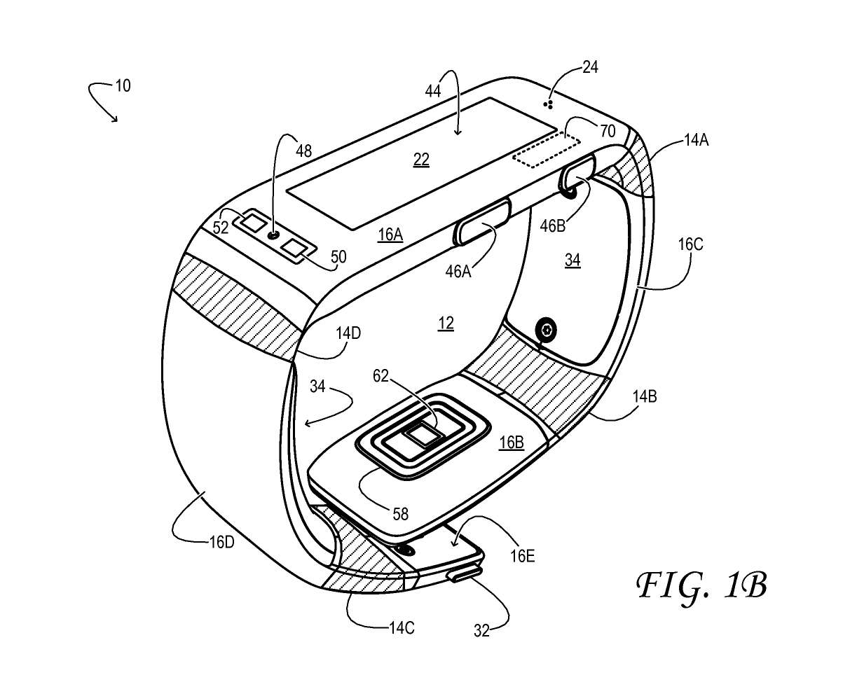

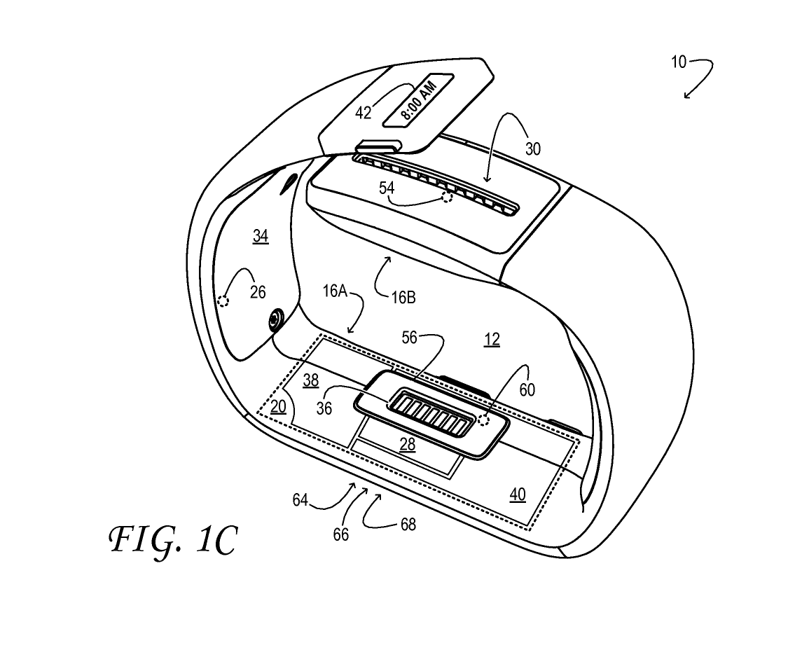

An optical pulse-rate sensor will now be described in the context of a wearable electronic device. It will be understood, however, that pulse-rate sensors as described herein may be incorporated in other devices as well, without departing from the scope of this disclosure. FIGS. 1A-C show aspects of a wearable electronic device 10 in one, non-limiting configuration. The illustrated device takes the form of a composite band 12, which may be worn around a wrist. Composite band 12 includes flexible segments 14 and rigid segments 16. The terms ‘flexible’ and ‘rigid’ are to be understood in relation to each other, not necessarily in an absolute sense. Moreover, a flexible segment may be relatively flexible with respect to one bending mode and/or stretching mode, while being relatively inflexible with respect to other bending modes, and to twisting modes. A flexible segment may be elastomeric in some examples. In these and other examples, a flexible segment may include a hinge and may rely on the hinge for flexibility, at least in part.

The illustrated configuration includes four flexible segments 14 linking five rigid segments 16. Other configurations may include more or fewer flexible segments, and more or fewer rigid segments. In some implementations, a flexible segment is coupled between pairs of adjacent rigid segments.

Various functional components, sensors, energy-storage cells, etc., of wearable electronic device 10 may be distributed among multiple rigid segments 16. Accordingly, as shown schematically in FIG. 1A, one or more of the intervening flexible segments 14 may include a course of electrical conductors 18 running between adjacent rigid segments, inside or through the intervening flexible segment. The course of electrical conductors may include conductors that distribute power, receive or transmit a communication signal, or carry a control or sensory signal from one functional component of the device to another. In some implementations, a course of electrical conductors may be provided in the form of a flexible printed-circuit assembly (FPCA, vide infra), which also may physically support various electronic and/or logic components.

In one implementation, a closure mechanism enables facile attachment and separation of the ends of composite band 12, so that the band can be closed into a loop and worn on the wrist. In other implementations, the device may be fabricated as a continuous loop resilient enough to be pulled over the hand and still conform to the wrist. In still other implementations, wearable electronic devices of a more elongate band shape may be worn around the user's bicep, waist, chest, ankle, leg, head, or other body part. Accordingly, the wearable electronic devices here contemplated include eye glasses, a head band, an arm-band, an ankle band, a chest strap, or even an implantable device to be implanted in tissue.

As shown in FIGS. 1B and 1C, wearable electronic device 10 includes various functional components: a compute system 20, display 22, loudspeaker 24, haptic motor 26, communication suite 28, and various sensors. In the illustrated implementation, the functional components are integrated into rigid segments 16—viz., display-carrier module 16A, pillow 16B, battery compartments 16C and 16D, and buckle 16E. This tactic protects the functional components from physical stress, from excess heat and humidity, and from exposure to water and substances found on the skin, such as sweat, lotions, salves, and the like.

In the illustrated conformation of wearable electronic device 10, one end of composite band 12 overlaps the other end. A buckle 16E is arranged at the overlapping end of the composite band, and a receiving slot 30 is arranged at the overlapped end. As shown in greater detail herein, the receiving slot has a concealed rack feature, and the buckle includes a set of pawls to engage the rack feature. The buckle snaps into the receiving slot and slides forward or backward for proper adjustment. When the buckle is pushed into the slot at an appropriate angle, the pawls ratchet into tighter fitting set points. When release buttons 32 are squeezed simultaneously, the pawls release from the rack feature, allowing the composite band to be loosened or removed.

The functional components of wearable electronic device 10 draw power from one or more energy-storage cells 34. A battery—e.g., a lithium ion battery—is one type of energy-storage cell suitable for this purpose. Examples of alternative energy-storage cells include super- and ultra-capacitors. A typical energy storage cell is a rigid structure of a size that scales with storage capacity. To provide adequate storage capacity with minimal rigid bulk, a plurality of discrete separated energy storage cells may be used. These may be arranged in battery compartments 16C and 16D, or in any of the rigid segments 16 of composite band 12. Electrical connections between the energy storage cells and the functional components are routed through flexible segments 14. In some implementations, the energy storage cells have a curved shape to fit comfortably around the wearer's wrist, or other body part.

In general, energy-storage cells 34 may be replaceable and/or rechargeable. In some examples, recharge power may be provided through a universal serial bus (USB) port 36, which includes a magnetic latch to releasably secure a complementary USB connector. In other examples, the energy storage cells may be recharged by wireless inductive or ambient-light charging. In still other examples, the wearable electronic device may include electro-mechanical componentry to recharge the energy storage cells from the user's adventitious or purposeful body motion. More specifically, the energy-storage cells may be charged by an electromechanical generator integrated into wearable electronic device 10. The generator may be actuated by a mechanical armature that moves when the user is moving.

In wearable electronic device 10, compute system 20 is housed in display-carrier module 16A and situated below display 22. The compute system is operatively coupled to display 22, loudspeaker 24, communication suite 28, and to the various sensors. The compute system includes a data-storage machine 38 to hold data and instructions, and a logic machine 40 to execute the instructions.

Display 22 may be any suitable type of display, such as a thin, low-power light emitting diode (LED) array or a liquid-crystal display (LCD) array. Quantum-dot display technology may also be used. Suitable LED arrays include organic LED (OLED) or active matrix OLED arrays, among others. An LCD array may be actively backlit. However, some types of LCD arrays—e.g., a liquid crystal on silicon, LCOS array—may be front-lit via ambient light. Although the drawings show a substantially flat display surface, this aspect is by no means necessary, for curved display surfaces may also be used. In some use scenarios, wearable electronic device 10 may be worn with display 22 on the front of the wearer's wrist, like a conventional wristwatch. However, positioning the display on the back of the wrist may provide greater privacy and ease of touch input. To accommodate use scenarios in which the device is worn with the display on the back of the wrist, an auxiliary display module 42 may be included on the rigid segment opposite display-carrier module 16A. The auxiliary display module may show the time of day, for example.

Communication suite 28 may include any appropriate wired or wireless communications componentry. In FIGS. 1B and 1C, the communications suite includes USB port 36, which may be used for exchanging data between wearable electronic device 10 and other computer systems, as well as providing recharge power. The communication suite may further include two-way Bluetooth, Wi-Fi, cellular, near-field communication, and/or other radios. In some implementations, the communication suite may include an additional transceiver for optical, line-of-sight (e.g., infrared) communication.

In wearable electronic device 10, touch-screen sensor 44 is coupled to display 22 and configured to receive touch input from the user. Accordingly, the display may be a touch-sensor display in some implementations. In general, the touch sensor may be resistive, capacitive, or optically based. Push-button sensors (e.g., microswitches) may be used to detect the state of push buttons 46A and 46B, which may include rockers. Input from the push-button sensors may be used to enact a home-key or on-off feature, control audio volume, microphone, etc.

FIGS. 1B and 1C show various other sensors of wearable electronic device 10. Such sensors include microphone 48, visible-light sensor 50, ultraviolet sensor 52, and ambient-temperature sensor 54. The microphone provides input to compute system 20 that may be used to measure the ambient sound level or receive voice commands from the user. Input from the visible-light sensor, ultraviolet sensor, and ambient-temperature sensor may be used to assess aspects of the user's environment. In particular, the visible-light sensor can be used to sense the overall lighting level, while the ultraviolet sensor senses whether the device is situated indoors or outdoors. In some scenarios, output from the visible light sensor may be used to automatically adjust the brightness level of display 22, or to improve the accuracy of the ultraviolet sensor. In the illustrated configuration, the ambient-temperature sensor takes the form a thermistor, which is arranged behind a metallic enclosure of pillow 16B, next to receiving slot 30. This location provides a direct conductive path to the ambient air, while protecting the sensor from moisture and other environmental effects.

FIGS. 1B and 1C show a pair of contact sensors—charging contact sensor 56 arranged on display-carrier module 16A, and pillow contact sensor 58 arranged on pillow 16B. Each contact sensor contacts the wearer's skin when wearable electronic device 10 is worn. The contact sensors may include independent or cooperating sensor elements, to provide a plurality of sensory functions. For example, the contact sensors may provide an electrical resistance and/or capacitance sensory function responsive to the electrical resistance and/or capacitance of the wearer's skin. To this end, the two contact sensors may be configured as a galvanic skin-response sensor, for example. Compute system 20 may use the sensory input from the contact sensors to assess whether, or how tightly, the device is being worn, for example. In the illustrated configuration, the separation between the two contact sensors provides a relatively long electrical path length, for more accurate measurement of skin resistance. In some examples, a contact sensor may also provide measurement of the wearer's skin temperature. In the illustrated configuration, a skin temperature sensor 60 in the form a thermistor is integrated into charging contact sensor 56, which provides direct thermal conductive path to the skin. Output from ambient-temperature sensor 54 and skin temperature sensor 60 may be applied differentially to estimate of the heat flux from the wearer's body. This metric can be used to improve the accuracy of pedometer-based calorie counting, for example. In addition to the contact-based skin sensors described above, various types of non-contact skin sensors may also be included.

Arranged inside pillow contact sensor 58 in the illustrated configuration is an optical pulse-rate sensor 62. The optical pulse-rate sensor may include a narrow-band (e.g., green) LED emitter and matched photodiode to detect pulsating blood flow through the capillaries of the skin, and thereby provide a measurement of the wearer's pulse rate. In some implementations, the optical pulse-rate sensor may also be configured to sense the wearer's blood pressure. In the illustrated configuration, optical pulse-rate sensor 62 and display 22 are arranged on opposite sides of the device as worn. The pulse-rate sensor alternatively could be positioned directly behind the display for ease of engineering. In some implementations, however, a better reading is obtained when the sensor is separated from the display.

Wearable electronic device 10 may also include motion sensing componentry, such as an accelerometer 64, gyroscope 66, and magnetometer 68. The accelerometer and gyroscope may furnish inertial data along three orthogonal axes as well as rotational data about the three axes, for a combined six degrees of freedom. This sensory data can be used to provide a pedometer/calorie-counting function, for example. Data from the accelerometer and gyroscope may be combined with geomagnetic data from the magnetometer to further define the inertial and rotational data in terms of geographic orientation.

Wearable electronic device 10 may also include a global positioning system (GPS) receiver 70 for determining the wearer's geographic location and/or velocity. In some configurations, the antenna of the GPS receiver may be relatively flexible and extend into flexible segment 14A. In the configuration of FIGS. 1B and 1C, the GPS receiver is far removed from optical pulse-rate sensor 62 to reduce interference from the optical pulse-rate sensor. More generally, various functional components of the wearable electronic device—display 22, compute system 20, GPS receiver 70, USB port 36, microphone 48, visible-light sensor 50, ultraviolet sensor 52, and skin temperature sensor 60—may be located in the same rigid segment for ease of engineering, but the optical pulse-rate sensor may be located elsewhere to reduce interference on the other functional components.

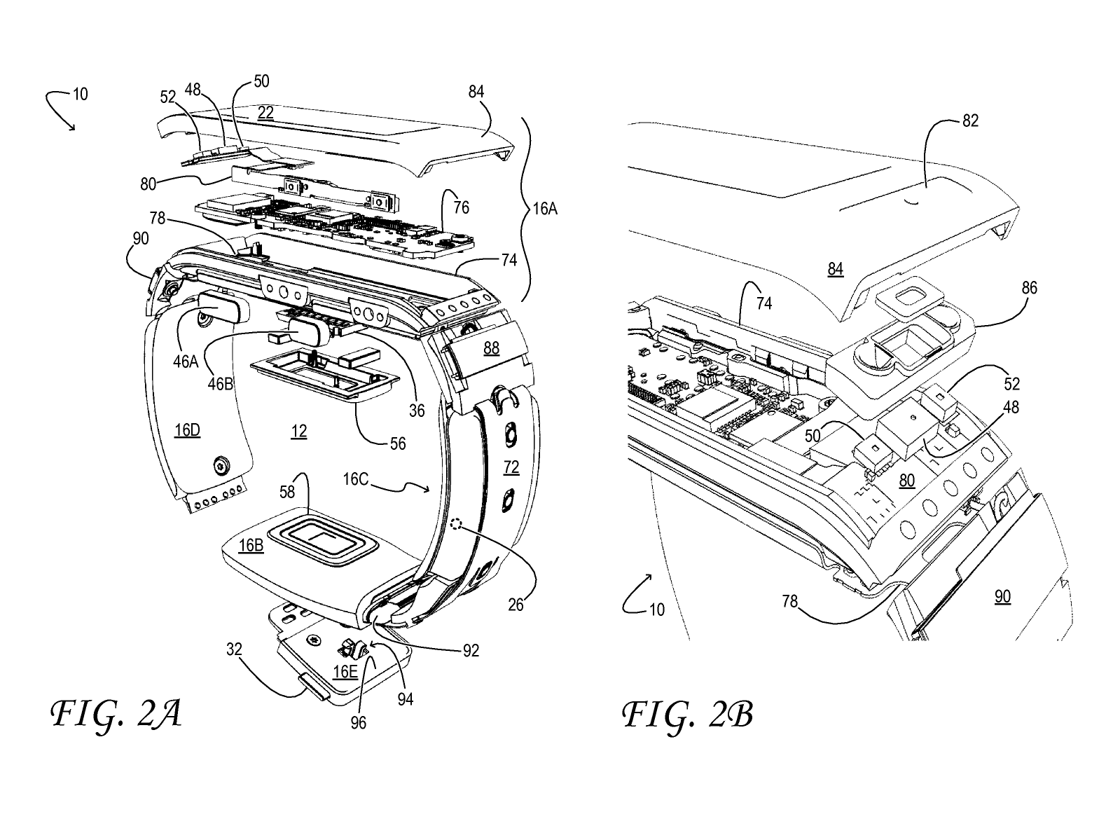

FIGS. 2A and 2B show aspects of the internal structure of wearable electronic device 10 in one, non-limiting configuration. In particular, FIG. 2A shows semi-flexible armature 72 and display carrier 74. The semi-flexible armature is the backbone of composite band 12, which supports display-carrier module 16A, pillow 16B, and battery compartments 16B and 16C. The semi-flexible armature may be a very thin band of steel, in one implementation. The display carrier may be a metal frame overmolded with plastic. It may be attached to the semi-flexible armature with mechanical fasteners. In one implementation, these fasteners are molded-in rivet features, but screws or other fasteners may be used instead. The display carrier provides suitable stiffness in display-carrier module 16A to protect display 22 from bending or twisting moments that could dislodge or break it. In the illustrated configuration, the display carrier also surrounds the main printed circuit assembly (PCA) 76, where compute system 20 is located, and provides mounting features for the main PCA.

In some implementations, wearable electronic device 10 includes a main flexible FPCA 78, which runs from pillow 16B all the way to battery compartment 16D. In the illustrated configuration, the main FPCA is located beneath semi-flexible armature 72 and assembled onto integral features of the display carrier. In the configuration of FIG. 2A, push buttons 46A and 46B penetrate one side of display carrier 74. These push buttons are assembled directly into the display carrier and are sealed by o-rings. The push buttons act against microswitches mounted to sensor FPCA 80.

Display-carrier module 16A also encloses sensor FPCA 80. At one end of rigid segment 16A, and located on the sensor FPCA, are visible-light sensor 50, ultraviolet sensor 52, and microphone 48. A polymethylmethacrylate window 82 is insert molded into a glass insert-molded (GIM) bezel 84 of display-carrier module 16A, over these three sensors. The window has a hole for the microphone and is printed with IR transparent ink on the inside covering except over the ultraviolet sensor. A water repellent gasket 86 is positioned over the microphone, and a thermoplastic elastomer (TPE) boot surrounds all three components. The purpose of the boot is to acoustically seal the microphone and make the area more cosmetically appealing when viewed from the outside.

As noted above, display carrier 74 may be overmolded with plastic. This overmolding does several things. First, the overmolding provides a surface that the device TPE overmolding will bond to chemically. Second, it creates a shut-off surface, so that when the device is overmolded with TPE, the TPE will not ingress into the display carrier compartment. Finally, the PC overmolding creates a glue land for attaching the upper portion of display-carrier module 16A.

The charging contacts of USB port 36 are overmolded into a plastic substrate and reflow soldered to main FPCA 78. The main FPCA may be attached to the inside surface of semi-flexible armature 72. In the illustrated configuration, charging contact sensor 56 is frame-shaped and surrounds the charging contacts. It is attached to the semi-flexible armature directly under display carrier 74—e.g., with rivet features. Skin temperature sensor 60 (not shown in FIGS. 2A or 2B) is attached to the main FPCA under the charging contact-sensor frame, and thermal conduction is maintained from the frame to the sensor with thermally conductive putty.

FIGS. 2A and 2B also show a Bluetooth antenna 88 and a GPS antenna 90, which are coupled to their respective radios via shielded connections. Each antenna is attached to semi-flexible armature 72 on either side of display carrier 74. The semi-flexible armature may serve as a ground plane for the antennas, in some implementations. Formed as FPCAs and attached to plastic antenna substrates with adhesive, the Bluetooth and GPS antennas extend into flexible segments 14A and 14D, respectively. The plastic antenna substrates maintain about a 2-millimeter spacing between the semi-flexible armature and the antennae, in some examples. The antenna substrates may be attached to semi-flexible armature 72 with heat staked posts. TPE filler parts are attached around the antenna substrates. These TPE filler parts may prevent TPE defects like ‘sink’ when the device is overmolded with TPE.

Shown also in FIG. 2A are a metallic battery compartments 16C and 16D, attached to the inside surface of semi-flexible armature 72, such that main FPCA 78 is sandwiched between the battery compartments and the semi-flexible armature. The battery compartments have an overmolded rim that serves the same functions as the plastic overmolding previously described for display carrier 74. The battery compartments may be attached with integral rivet features molded-in. In the illustrated configuration, battery compartment 16C also encloses haptic motor 26.

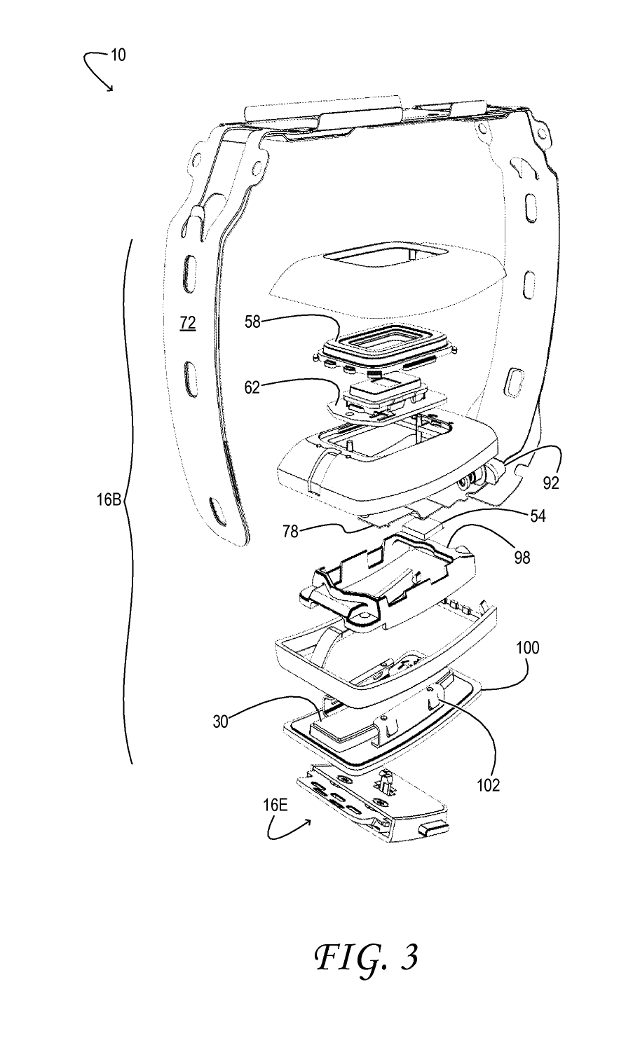

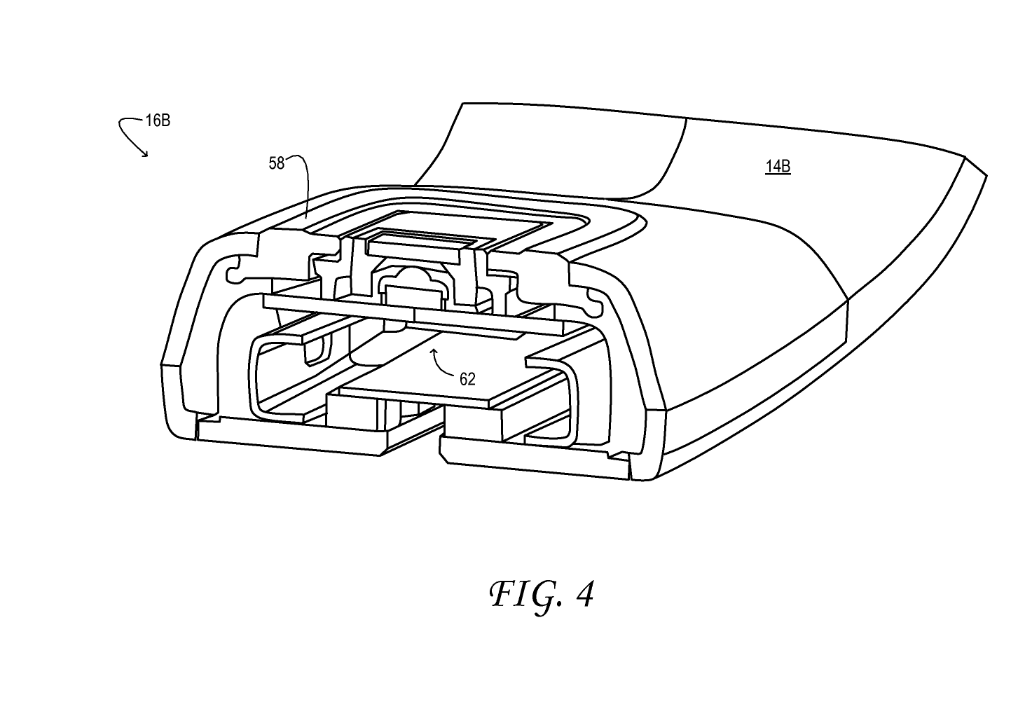

Shown also in FIG. 2A, a bulkhead 92 is arranged at and welded to one end of semi-flexible armature 72. This feature is shown in greater detail in the exploded view of FIG. 3. The bulkhead provides an attachment point for pillow contact sensor 58. The other end of the semi-flexible armature extends through battery compartment 16D, where flexible strap 14C is attached. The strap is omitted from FIG. 2 for clarity, but is shown in FIGS. 1B and 1C. In one example, the strap is attached with rivets formed integrally in the battery compartment. In another embodiment, a plastic end part of the strap is molded-in as part of the battery compartment overmolding process.

In the configuration of FIG. 2A, buckle 16E is attached to the other end of strap 14C. The buckle includes two opposing, spring-loaded pawls 94 constrained to move laterally in a sheet-metal spring box 96. The pawls and spring box are concealed by the buckle housing and cover, which also have attachment features for the strap. The two release buttons 32 protrude from opposite sides of the buckle housing. When these buttons are depressed simultaneously, they release the pawls from the track of receiving slot 30 (as shown in FIG. 1C).

Turning now to FIG. 3, pillow 16B includes pillow contact sensor 58, which surrounds optical pulse-rate sensor 62. The pillow also includes TPE and plastic overmoldings, an internal structural pillow case 98, and a sheet-metal or MIMS inner band 100. The pillow assembly is attached to bulkhead 92 with adhesives for sealing out water and by two screws that clamp the pillow case and the plastic overmolding securely to the bulkhead. The inner band includes receiving slot 30 and its concealed rack feature. In the illustrated configuration, the inner band is attached to the pillow via adhesives for water sealing and spring steel snaps 102, which are welded to the inside of the inner band on either side of the concealed rack. Main FPCA 78 extends through the bulkhead and into the pillow assembly, to pillow contact sensor 58. Ambient-temperature sensor 54 is attached to this FPCA and surrounded by a small plastic frame. The frame contains thermal putty to help maintain a conduction path through the inner band to the sensor. On the opposite side of the FPCA from the sensor a foam spring may be used to push the sensor, its frame, and thermal putty against the inside surface of the inner band.

The foregoing drawings and description will help the reader to appreciate one of the many possible environments for optical pulse-rate sensor 62—viz., wearable electronic device 10. Additional aspects of the optical-pulse rate sensor are described below, with continued reference to wearable electronic device 10. It will be understood, however, that optical pulse-rate sensors in other, quite different environments lie fully within the spirit and scope of this disclosure. For instance, an optical pulse-rate sensor as described herein may be incorporated into headphones, such as ear buds, or held against virtually any part of the body using an adhesive strip or fully flexible band.

As noted above, pillow 16B is a fixture for various internal sensory components of wearable electronic device 10, including optical pulse-rate sensor 62. FIG. 4 provides a cross-sectional view of the pillow and optical pulse-rate sensor in one, non-limiting configuration. The pillow includes a protruding rim in the form of pillow contact sensor 58. When wearable electronic device 10 is worn by a user, the rim is substantially sealed against the user's skin, which limits ambient light from reaching the internal components of the optical pulse-rate sensor. In this manner, a potential noise source for the pulse measurement is greatly reduced. It will be noted that the ambient light-blocking rim structure of pillow contact sensor 58 is independent of the sensory function of this component (vide supra). Other implementations may include a rim having no sensory function per se.

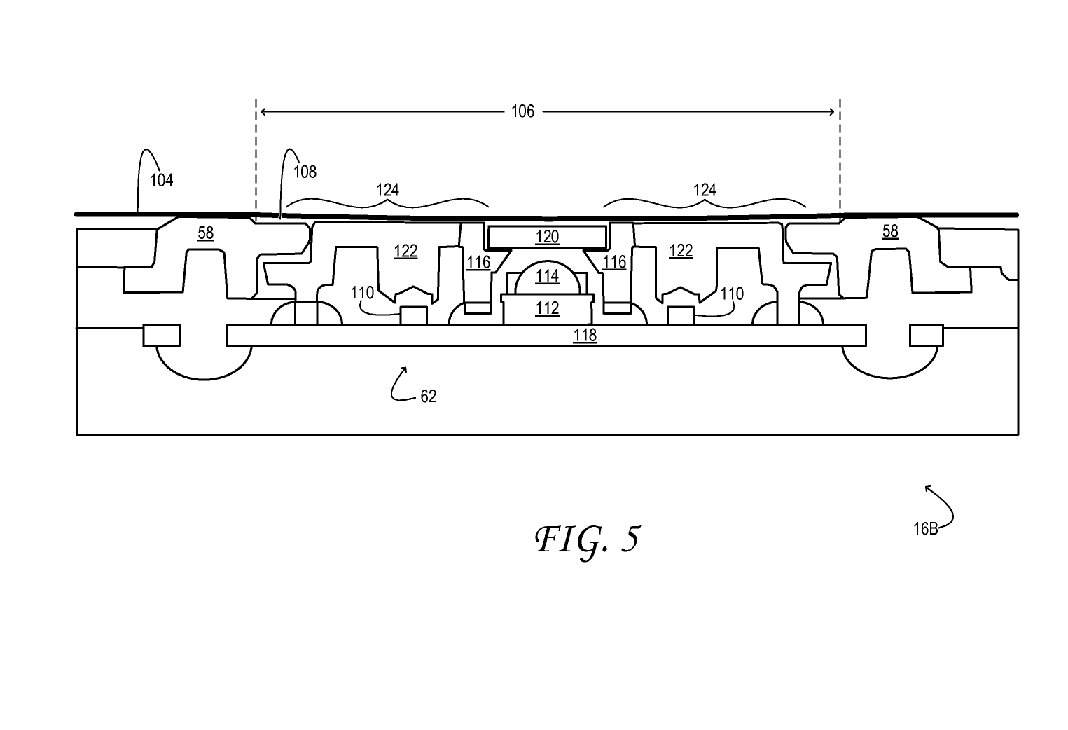

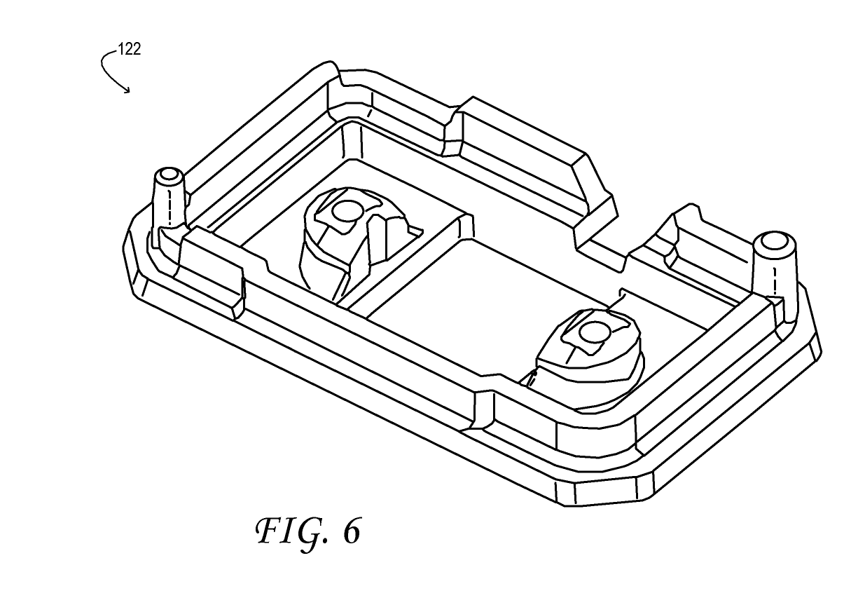

FIG. 5 provides another cross-sectional view of pillow 16B and optical pulse-rate sensor 62. As shown in this drawing, pillow contact sensor 58 is configured to contact a skin surface 104 of the wearer of wearable electronic device 10, and to enclose an area 106 of that surface. This is the area of skin through which the wearer's pulse rate is to be measured. As described hereinabove, optical pulse-rate sensor 62 may be integrated into a composite band 12 (of FIGS. 1A and 1B), which is connected to the pillow and configured to press the pillow contact sensor against the skin surface when the wearable electronic device is worn.

In the illustrated example, optical pulse-rate sensor 62 includes a pair of light emitters 110 coupled to pillow 16B and positioned opposite area 106. A light sensor 112 is also coupled to this fixture and positioned opposite the area. In the illustrated configuration, a hemispherical lens 114 is positioned over the light sensor to increase the amount of light from area 106 that is received into the acceptance cone of the light sensor. By placing this lens directly on the light sensor—the lens having a diameter that closely matches the width and height of the light sensor—improved collection efficiency is achieved. In particular, the effective area of the light sensor is increased by a factor equal to the magnification of the lens. In some examples, the lens is formed as a separate molded part or as a precise droplet of UV curable optical adhesive. In other examples, the lens may be molded into the clear plastic package of the light sensor.

The principle of operation of optical pulse-rate sensor 62 is the attenuance of visible light by hemoglobin in the wearer's blood, which flows behind skin surface 104. With each contraction of the heart muscle, capillaries close to the skin surface are charged with blood. With each relaxation between successive contractions, the capillaries are partially emptied. Thus, the skin and the tissue beneath the skin surface will contain more hemoglobin per unit volume during a contraction than during a relaxation. This layer of tissue is probed with visible light from light emitters 110. The light is reflected from, but also penetrates the skin to a significant thickness. The penetrating light is subject to repeated scattering in the tissue, and to absorption by the hemoglobin, as it passes through the capillaries. Some of the penetrating light will be scattered out of the skin through area 106. This light will be attenuated to a greater degree during a contraction of the heart muscle than during a relaxation, due to the changing amount of hemoglobin in the tissue, according to the Beer-Lambert law. A plot of the light intensity received at light sensor 112 is a periodic function, therefore, with a frequency equal to the wearer's pulse rate. An analog-to-digital converter arranged on pillow PCA 118 or TDM 16A digitizes the output from the light sensor, and provides such output to compute system 20, which computes the wearer's pulse rate based on the digitized periodic output of the light sensor. In some implementations, the bias to the light emitters may be modulated, and a lock-in detection scheme may be used to improve signal-to-noise in the pulse-rate determination.

In the implementation illustrated in FIG. 5, optical pulse-rate sensor 62 includes a recess portion 108 inside the rim, which reduces contact pressure on area 106 when the rim is in contact with skin surface 104. This feature may help to avoid a ‘bleaching’ effect, where excessive contact pressure hinders the refill of blood into the capillaries directly above area 106, causing a reduction in signal. Thus, the recess portion serves both to improve signal recovery times by allowing blood to re-enter bleached skin more quickly, and to prevent bleaching-based signal loss. In this manner, the recess portion can make the sensor more accurate, especially when the user is exercising vigorously, such that movement of the device on the skin is more likely to occur. In some configurations and use scenarios, recess portion 108 is low enough to escape contact with skin surface 104, thereby preventing any reduction in signal due to bleaching. In other configurations, the recess portion may be higher, so that the skin surface is contacted in area 106, but with less pressure. In still other configurations, the recess portion may be omitted entirely, so that the optical pulse-rate sensor profile is substantially flat.

The rim and recess portion 108, if included, may be formed in any suitable manner. In the illustrated configuration, pillow contact sensor 58 (the rim) has a slight step in its outer surface (the surface that contacts the wearer's skin). As such, the outer most surface of the pillow contact sensor is higher than the inner surface of the pillow contact sensor, and higher than the recessed componentry of optical pulse-rate sensor 62, which the pillow contact sensor circumscribes.

In the configuration of FIG. 5, optical pulse-rate sensor 62 also includes light stop 116. The light stop is coupled to pillow 16B and positioned between light emitters 110 and light sensor 112. The purpose of the light stop is to shield the light sensor and lens from direct illumination by the light source, for increased signal-to-noise.

To reduce power consumption in optical pulse-rate sensor 62, each light emitter 110 may be a high-efficiency, narrow-band light emitting diode (LED). In particular, green LEDs may be used, whose emission closely matches the absorption maximum of hemoglobin. Various numbers and arrangements of light emitters may be used without departing from the scope of this disclosure. The illustrated example shows two light emitters arranged symmetrically on opposite sides of light sensor 112.

In one implementation, light sensor 112 may be a photodiode. In other implementations, a phototransistor or other type of light sensor may be used. In the configuration shown in FIG. 5, light emitters 110 and light sensor 112 are coupled to pillow PCA 118. The pillow PCA may also include electronics configured to drive the light emitter, receive output from the light sensor, and based on the output, to generate data responsive to a pulse rate of blood flowing under the skin surface. In other implementations, at least some of the electronics may be situated elsewhere—in display carrier module 16A, for example—or distributed between the pillow PCA and any other fixture on the device.

In the configuration of FIG. 5, an optical filter 120 is positioned over light sensor 112 and lens 114 to limit the wavelength range of light received into the light sensor. In the illustrated configuration, light stop 116 is shaped to seat the optical filter. The optical filter may be configured to transmit light in the emission band of light emitters 110, but to block light of other wavelengths, such as broadband ambient light that may leak under the rim. In some implementations, the optical filter is a band-pass filter with a pass band matched to the emission band of the light emitters. The optical filter may be a dichroic filter in one implementation. The use of a dichroic filter offers a manufacturing advantage over an absorbing filter. In particular, a dichroic filter can be attached using an ultraviolet (UV) curable glue. UV light can pass through the dichroic filter where the glue is applied and not be attenuated. By using a dichroic filter, a very narrow pass band can be achieved, while simultaneously curing with light of a wavelength range outside the pass band of the filter. As the function of the dichroic is dependent on an air gap, it is possible to cure with light outside the pass band, in contrast to an absorbing filter. In another implementation, the optical filter may be another type of non-absorbing interference filter, or, a holographic filter which discriminates according to angle of the light received in addition to wavelength.

The illustrated optical pulse-rate sensor 62 also includes a light guide 122. The light guide is configured to collect the angle-distributed emission from light emitters 110 and redirect the emission towards skin surface 104. The light guide is further configured to disperse the emission to substantially cover area 106. FIG. 6 shows aspects of an example light guide 122 in one, non-limiting configuration. The isometric view of FIG. 6 is from the point of view of pillow PCA 118 (of FIG. 5).

Light guide 122 may be fabricated from any suitable transparent polymer, such as polyacrylic. The light guide may be surrounded by air or by a cladding of a lower refractive index than the polymer from which the light guide is fabricated. Accordingly, the light guide may be configured to redirect and disperse collected emission via total internal reflection. Through repeated internal reflections at the boundary surfaces of the light guide, the propagating light changes direction and diverges to all regions of area 106. In particular, the boundary edges of the light guide direct the light to spread out into regions of area 106 from which the unabsorbed portion will reflect directly into light sensor 112. This feature increases the signal-to-noise ratio of the optical pulse-rate measurement.

In one implementation, light stop 116 and light guide 122 may be formed in the same mold, to create a housing 124 that attaches to the PCA over the light emitters, lens, and light sensor. In one configuration, the housing includes two different plastics. The first is an optically opaque black plastic that surrounds the light sensor on four sides to form light stop 116. The rest of the housing may be made of a clear plastic, thus forming light guide 122. In one example, the composite housing is attached to pillow 16B with an optically opaque black glue. In another example, an optically clear glue may be used, or a die-cut adhesive. In these and other examples, an optically opaque black glue may be applied between light stop 116 and pillow PCA 118, for added light-blocking.

In one implementation, optical pulse-rate sensor 62 is sealed around its periphery and securely attached to pillow 16B. In one implementation, housing 124 is datumed through a hole in the pillow, and this joint is sealed with adhesive. In this and other implementations, two projections or posts from the pillow may extend through pillow PCA 118. These posts are subsequently heat staked so that a permanent mechanical attachment is attained.

Because it is not desirable for optical pulse-rate sensor 62 to be installed in wearable electronic device 10 while the device undergoes TPE overmolding, the pillow 16B may be constructed as a separate unit and attached to the device during final assembly after TPE overmolding. In order to make the required electrical connections, an extension of the main FPCA 78 is left extending from the device after overmolding. This FPCA extension is threaded through a hole at the juncture of the pillow assembly at the end of the device and accessed via a zero insertion-force (ZIF) connector. The outside of pillow 16B is finally closed by installing inner band 100.

The implementations described above should not be understood in a limiting sense, because numerous other implementations lie within the spirit and scope of this disclosure. For example, though the forgoing configurations show both the light emitters and the light sensor arranged opposite the surface of the skin where the optical pulse-rate measurement takes place, it is also envisaged that a light emitter may be positioned on one side of a skin layer (e.g., an earlobe, finger, or nasal septum), and a light sensor positioned on the opposite side of the skin layer. In other words, the optical pulse-rate measurement can be transmissive instead of reflective.

Compute system 20, via the sensory functions described herein, is configured to acquire various forms of information about the wearer of wearable electronic device 10. Such information must be acquired and used with utmost respect for the wearer's privacy. Accordingly, the sensory functions may be enacted subject to opt-in participation of the wearer. In implementations where personal data is collected on the device and transmitted to a remote system for processing, that data may be anonymized. In other examples, personal data may be confined to the wearable electronic device, and only non-personal, summary data transmitted to the remote system.

It will be understood that the configurations and approaches described herein are exemplary in nature, and that these specific implementations or examples are not to be taken in a limiting sense, because numerous variations are feasible. The specific routines or methods described herein may represent one or more processing strategies. As such, various acts shown or described may be performed in the sequence shown or described, in other sequences, in parallel, or omitted.

The subject matter of this disclosure includes all novel and non-obvious combinations and sub-combinations of the various processes, systems and configurations, and other features, functions, acts, and/or properties disclosed herein, as well as any and all equivalents thereof.

1. An optical pulse-rate sensor comprising:
a fixture with a rim configured to contact a skin surface and enclose an area of the surface;
a light emitter coupled to the fixture and positioned opposite the area;
a light sensor coupled to the fixture and positioned opposite the area;
a lens positioned over the light sensor; and
a light stop coupled to the fixture and positioned between the light emitter and the light sensor to shield the light sensor and lens from direct illumination by the light source.

2. The optical pulse-rate sensor of claim 1 wherein the light emitter includes a light emitting diode.

3. The optical pulse-rate sensor of claim 1 wherein the light emitter is one of a plurality of light emitters coupled to the fixture, positioned opposite the area.

4. The optical pulse-rate sensor of claim 1 wherein the light sensor is a photodiode or phototransistor.

5. The optical pulse-rate sensor of claim 1 further comprising a recess portion inside the rim, which reduces contact pressure on the area when the rim is in contact with the skin surface.

6. The optical pulse-rate sensor of claim 5 wherein the recess portion does not contact the skin surface.

7. The optical pulse-rate sensor of claim 1 further comprising an optical filter positioned over the light sensor to limit a wavelength range of light received into the light sensor.

8. The optical pulse-rate sensor of claim 7 wherein the light stop is configured to seat the optical filter.

9. The optical pulse-rate sensor of claim 7 wherein the optical filter is a band-pass filter.

10. The optical pulse-rate sensor of claim 7 wherein the optical filter is a dichroic filter.

11. The optical pulse-rate sensor of claim 7 wherein the optical filter is a holographic filter.

12. The optical pulse-rate sensor of claim 1 wherein emission of the light emitter is limited to a narrow visible wavelength band.

13. The optical pulse-rate sensor of claim 1 wherein the fixture is configured to prevent ambient light from reaching the light sensor.

14. A wearable electronic device comprising:
a fixture with a rim configured to contact a skin surface and enclose an area of the surface;
a light emitter coupled to the fixture and positioned opposite the area;
a light sensor coupled to the fixture and positioned opposite the area;
an interference filter positioned over the light sensor to limit a wavelength range of light received into the light sensor;
electronics configured to drive the light emitter, receive output from the light sensor, and based on the output, to generate data responsive to a pulse rate of blood flowing under the skin surface; and
a band connected to the fixture and configured to press the rim against the skin surface when the wearable electronic device is worn.

15. The wearable electronic device of claim 14 wherein the fixture is a first fixture, and wherein the electronics configured to generate the output data is arranged in a second fixture separated from the first fixture by at least one flexible segments of the band.

16. The wearable electronic device of claim 14 wherein the band includes a flexible printed circuit assembly to which the fixture is electronically coupled.

17. An optical pulse-rate sensor comprising:
a fixture with a rim configured to contact a skin surface and enclose an area of the surface;
a light emitter coupled to the fixture and positioned opposite the area;
a light sensor coupled to the fixture and positioned opposite the area;
a light stop coupled to the fixture and positioned between the light emitter and the light sensor to shield the light sensor from direct illumination by the light emitter; and
a light guide configured to collect light from the light emitter and redirect the light towards the surface.

18. The optical pulse-rate sensor of claim 17 wherein the light guide is further configured to disperse the light to substantially cover the area.

19. The optical pulse-rate sensor of claim 17 wherein the light guide redirects the light at least partly by total internal reflection of the light.

20. The optical pulse-rate sensor of claim 17 wherein the light stop and the light guide are formed in the same mold.
